(19) United States
(12) Patent Application Publication
(10) Pub. No.: US 2007/0189979 A1
(43) Pub. Date: Aug. 16, 2007

(54) Particles

(75) Inventors: Xian-Ming Zeng, Mitcham (GB); Austen John Woolfe, North Weald (GB); Alan Langford, Hoddesdon (GB)

Correspondence Address: LERNER, DAVID, LITTENBERG,;KRUMHOLZ & MENTLIK, 600 SOUTH AVENUE WEST, WESTFIELD, NJ, 07090, US

(73) Assignee: Norton Healthcare Limited t/a IVAX Pharmaceuticals, London (GB)

(21) Appl. No.: 10/572,754

(22) PCT Filed: Sep. 18, 2004
(86) PCT No.: PCT/US04/30724

§ 371 Date: Apr. 11, 2007

(30) Foreign Application Priority Data
Sep. 18, 2003 (GB) 0321873.2
Feb. 13, 2004 (GB) 0403262.9

(51) Int. Cl. A61K 31/573; A61K 31/353; A61K 9/14

(52) U.S. Cl. 424/46; 424/490; 514/171; 514/454

(57) ABSTRACT

The invention relates to particles for drug delivery by inhalation, said particles incorporating at least one active ingredient which is non-crystalline. The may be a plurality of active ingredients and moreover the outer surface of the particles may be substantially smooth. By providing non-crystalline particles with an outer smooth surface, a substantially accurate dose of active ingredient(s) can, in use of an inhalation device, be delivered each time the device is discharged, with a free flow and non-agglomeration of the particles. This is brought about by the smooth surface and the lack of a periodic ordered structure typical of a crystalline solid. The invention extends to an inhalation composition, and a pulmonary nasal inhalation device including such a composition.

### CROSS REFERENCE TO RELATED APPLICATIONS

This application claims the benefit of priority of Provisional Application No. filed and Provisional Application No. filed both of which are incorporated herein by reference.

### FIELD OF THE INVENTION

The invention relates to particles, particularly to particles which may include at least one active ingredient and which are suitable for inhalation and for the provision of inhalation compositions.

Such particles and compositions are particularly suitable for treatment of pulmonary disorders such as asthma, and as such are, it will be understood, suitable for pulmonary drug delivery. These particles and compositions may also be suitable for systemic absorption from lungs as a preferred route into the blood stream.

### BACKGROUND OF THE INVENTION

It is generally accepted that inhalation is a proven route for treatment of asthma. A drug or drugs being administered by inhalation, which can be oral or nasal, have a direct route from an inhaler device to the lungs of the user of the device, so providing rapid action.

Known particles and inhalant compositions incorporating same, however, often comprise particles having a generally rough external surface. This leads to agglomeration as the particles tend to lock physically together with each other and with any solid carrier excipient which may be present including lactose in aerosols or dry powder inhalers, and thus agglomerate into clumps of particles. These dumps of particles have less than optimal aerodynamic size for effective penetration into the deep lung. This can lead also to an irregular or non-prescribed amount of active ingredient being supplied in a single delivery and moreover, the dumps can block a discharge orifice of an inhalation device.

It is an object of the invention to seek to mitigate these disadvantages.

### DETAILED DESCRIPTION OF THE INVENTION

According to a first aspect of the invention there is provided particles for drug delivery by inhalation, which particles incorporate at least one active ingredient which is non-crystalline.

Using the invention it is possible to accurately deliver drugs which are more commonly used in aerosols and dry powder inhalers and which are delivered at very low doses. Thus a drug such as formoterol fumarate is often delivered at about 6 micrograms per dose. The use of particles embodying the invention seeks to provide both a more uniform loading of such small quantities of such drugs in dry powder inhalers and to prevent any content uniformity problems traditionally caused by the differences in density of two or more active ingredients in suspension aerosols. Moreover, use of particles embodying the invention may also obviate dumping or deposition onto walls of an aerosol can, and in metered-dose powder inhalers (MDPI) may provide for lower forces of adherence between the particles and the carrier excipient, for example lactose or mannitol. Under these conditions once any agglomerates of particles containing the active and carrier excipient enter the air stream they will generally break up readily to give a high fine particle fraction which is carried to the lungs and carrier excipient particles which lodge in the throat or buccal cavity.

The particles may contain a plurality of active ingredients, each of which may be non-crystalline. Suitably, the outer surface of the particles may be substantially smooth. It will be understood that the term “smooth” used herein means generally “lacking roughness”. The particles may suitably be spherical, for example the particles may be oblate spheroidal. Alternatively, the particles may be substantially oval or substantially elliptical. Suitably, the particles may have a particle size in the range 0.5 μm-5 μm, preferably 1 μm-3 μm; when substantially oval or elliptical, the longer axis of the particles may be 1-3 μm.

By providing non-crystalline particles with an outer smooth surface, a substantially accurate dose of active ingredient(s) can, in use of an inhalation device, be delivered each time the device is discharged, with a free flow and non-agglomeration of the particles. This is brought about by the smooth surface and the lack of a periodic ordered structure typical of a crystalline solid. Furthermore, when an excipient is included within the particles, the small particles of active ingredient are embedded within the excipient and in general do not come into contact with any moisture which may be present Once in the lungs, the active ingredients in the particles will be adsorbed more quickly because of their non-crystalline form.

Furthermore, as the particles are substantially smooth and spherical there is very little surface contact between the particles because of this generally spherical configuration. In addition, particle interlocking is substantially obviated, in contrast to such interlocking which would result from a rough surface of the particles. In dry powder inhalers where there are carrier particles such as lactose, where the lactose particles are much bigger than the particles of active ingredient, (say 60 microns carrier, to 1-2 microns active), then rough particles of active will stick more to the carrier particles than would smooth particles. It is preferable to minimise forces of adherence between the particles and the lactose or other carrier particles, so that when the formulation enters the air stream any agglomeration of particles and carrier will readily break up, increasing the fine particle fraction of active. As previously described higher fine particle fractions are desirable for efficacious pulmonary effect.

The particles may be electrically uncharged and may be provided by a method selected from the group comprising rapid expansion of solutions using a supercritical fluid technique, precipitation from gas saturated solutions, gas anti-solvent systems, aerosol solvent extraction systems, and spray drying processes.

According to a second aspect of the invention there is provided an inhalation composition, comprising particles which incorporate at least one active ingredient which is non-crystalline.

The particles may suitably contain a plurality of active ingredients, of which one at least may be non-crystalline. Thus for example there may be from one to four active ingredients in a particle of the composition which may also comprise a pharmaceutically acceptable particular excipient

The composition may in addition comprise a plurality of carrier excipient(s), which may comprise a modifier or stabiliser, or a chemical buffer, antioxidant and the like such as a surface modifier or surfactant Each carrier excipient takes up and holds the particles and assists in providing a consistent, accurate dispensing of an inhalation dose when the composition is dispensed from an inhalation device

The outer surface of the particles of the composition may preferably be substantially smooth, for advantageous discharge as hereinbefore described, which particles may be substantially spherical or oblate spheroidal. Alternatively, the particles may be substantially oval or substantially elliptical.

In the composition the particle size suitably may be in the range 0.5 pm-5 pm, and preferably 1 μm-3 μm. This provides for efficacy of treatment, particularly when the particles are electrically uncharged The particle size too helps to obviate any tendency to agglomeration of the particles of the composition.

Preferably, the active ingredients may comprise a β₂-agonist and a steroid or a β₂-agonist and an anticholinergic agent such as salmeterol xinafoate and tiotropium bronide. In a most preferred embodiment, the active ingredients may comprise a composition of fluticasone and salmeterol xinafoate, and/or formoterol and budesonide.

According to a third aspect of the invention there is provided an inhaler device comprising an inhalation composition as hereiebefore defined.

The excipient is preferably non-soluble in the propellant In aerosols, preferred propellants may be P134a, P227 or a mixture thereof.

It will be understood that particles embodying the invention may be produced to the preferred substantially spherical configuration in the particle size range 1 μm-3 μm diameter in any desired process including by a spray drying process, and that the end product particles containing from 1 or more, preferably up to 4 active ingredients, need no further processing. This is particularly so when a supercritical fluid technique is used to produce the particles.

The one or more active ingredients are in a constant desired ratio for a particular dose, and may be selected from β₂-agonists, steroids, anticholinergics or leucotrienes. The β-agonists may be short-or long-acting. Suitable steroids may be beclamethasone dipropionate, fluticasone etiprednol and budesonide.

The particle excipient(s) when used may suitably be water soluble and may bind the active ingredients when there are two or more such ingredients. Suitable water soluble excipient are PVP, Macrogel, Hydroxy Methyl Cellulose (MC), polyethylene glycol, mannitol, and poloxamer. The particle excipient must be able to be cleared from the lungs or nasal passages in a complete manner. Preferably, the main excipient is mannitol or PVP, and most preferably PVP (polyvinylpyrrolidone). Particles embodying the invention may be used alone or mixed with a carrier excipient such as lactose, or other suitable carrier, or in conjunction with P134A or P227 or a mixture thereof in an aerosol inhalation device together with other carrier excipient such as a solvent.

In one example, each particle comprises a mixture of two active ingredients, fluticasone and salmeterol xinafoate. In such a case:
a. Fluticasone is soluble to some extent in HFA propellants and ethanol and so the properties of the excipient in the particle must be to trap the fluticasone such that it cannot leach out in any moisture, or the aerosol propellant penetrate and dissolve the fluticasone out of the particle and into the liquid propellant.
b. To maintain the ratio of fluticasone and salmeterol xinafoate in the particle so that there is content uniformity; different ratios of the two drugs can be envisaged e.g. fluticasone 50-500 μg and salmeterol xinafoate 25 μg.
C. The choice of the excipient will be to meet the above two criteria, and at the same time have good suspension properties in typical aerosol formulation components. The suspended particles have physical properties such that suspension is easily maintained, and if the particles sediment or cream, then they are easily re-dispersed. They have very low affinity for the materials of the container, such as plastics and metals, and do not stick to the internal surfaces of the aerosol container.

In another example, the active ingredients may be formoterol and budesonide, in which case an excipient may not be required as the budesonide is present in a significantly greater amount than the formoterol e.g. in the ratio 100 micrograms: 6 micrograms or 400 micrograms: 12 micrograms.

Compositions suitable for nasal or pulmonary inhalation have been disclosed, suitable for example for treating asthma. Nevertheless particles embodying the invention may be used for all other suitable treatable conditions providing one or more appropriate active ingredients are used.

All particles and compositions embodying the invention generally include at least one active ingredient are stable and of a desired constant ratio of the active ingredients, where more than one active ingredient is/are used, and may preferably include a particle excipient There are advantages in utilising such a particle excipient (as opposed to a separate, carrier, excipient) which can be summarised as follows:

1) Protection from Moisture:

Formoterol embedded for example in polymer e.g. PVP. Formoterol is hydrolysed by moisture which may occur in a metered-dose inhaler (MDI) due to water ingress through ‘O’ ring seals etc, particularly if ethanol is present. Similarly in capsules for dry powder inhalation using metered-dose powder inhalers (MDPI) there is usually free moisture in the capsule shell which can transfer to the free drug, reducing flow and causing degradation. The use of polymer produces essentially non-crystalline, amorphous particles without hydroscopicity. Extra drugs can be added e.g. budesonide, and/or flow aids such as lubricants etc.
   2) Content Uniformity:
With a very low dose drug (e.g. formoterol where the normal dose is 6 micrograms per inhalation) and the drug is to be formulated with a second drug e.g. budesonide in a dry powder inhaler it is difficult particularly in a reservoir device to ensure that the correct doses of each individual drug are present in every dose. The use of preformed particles where the ratio of drugs is constant in every particle, ensures content uniformity in every dose. In an aerosol suspension formulation particularly with two or more drugs, the use of a combined particle will prevent content uniformity problems particularly where the drugs have differing densities which might otherwise entail differing dose uniformities due to the differences in density causing differences in suspendability (in the worst case, without the single particle, one drug could sink and one float).
   3) Solubility:
Where one drug is soluble in the propellant or propellant/co-solvent mixture, the use of a larger level of excipient than drug may enable, with control of drying parameters etc. embedding of the drug and if the excipient is insoluble in the propellant or propellant/co-solvent mixture, the drug will not dissolve. With two or more drugs where one is soluble and one or more is not, the use of a combined particle may again ensure a low variation in drug dose content uniformity.
   4) Stability:
The issue of stability against moisture has already been referred to above but additional carrier excipient e.g. buffers, antioxidants etc with the drug and polymer may stabilise a labile or pH sensitive drug, in other words when labile a drug which is unstable or liable to change. Both Formoterol and Budesonide are unstable in P134a and/or P134a/Ethanol mixtures. By embedding one or both in a propellant insoluble polymer a stable preparation can be obtained. In the case of Formoterol, a pH modifying agent may be added to the main excipient. A pH range of 2-8, preferably 2.5-6 and most preferably 3-5 is preferred.
   5) Adhesion:
Many drugs will adhere to the surfaces in an aerosol suspension particularly the wails and each other. The use of embedded particles, particularly if lubricants e.g. magnesium stearate or surfactants are added can ensure much better uniform resuspendability of the suspension
   6) Flow.
The use of additional excipient e.g. magnesium stearate etc may increase the flow characteristics of the particles and ensure a higher fine particle fraction when measured on a twin impinger or other impacter type device. This is particularly true if the device/capsule etc does not contain any other component except for the particles of the invention.
   7) Elimination of Unwanted Physiological Responses:
An instantaneous cough may be eliminated by embedding a cannabinol e.g. delta-9 THC in a particle of the invention prior to formulation in an MDI or MDPI. Similarly delta-8 THC or a mixture of delta-9 and delta-8 THC may be employed. Delta-9 THC is a delta tetrahydrocannabinol. Delta-8 THC is a derivative of the delta-9 molecule, and possesses similar properties. Delta-9 THC and its derivatives, including delta-8 THC, collectively cannabis, are known as cannabinoids. Such particles, which include cannabinoids, may have a rough surface.
   8) Bulklng Agent
Production of a larger weight of particles containing a small amount of drug or drugs will enable the dispersion of the drug much more easily both in aerosols and dry powder inhalers. For example the standard 6 or 12 microgrammes per dose of formoterol or 25 microgramies per dose of salmeterol can be relatively readily dispensed. These benefits are not necessarily single in nature. For example a particle containing delta-9 THC and PVP may prevent instantaneous cough from pure delta-9 THC but will also give a flowable particle in an MDPI whereas the drug itself is a very sticky oil with very poor flow characteristics. A particle containing formoterol may decrease the adherence to can walls in an MDI as well as giving a much improved stability. It will be understood that the term non-crystalline used herein includes particles or an active ingredient thereof which is amorphous. The active ingredient(s) may be amorphous, sticky or oily.

### EXAMPLES

### Example 1

Particles produced with Mannitol by Spray Drying.

| | |
|---|---|
| Solution concentration | 1.5% (w/v) in purified water, |
| Inlet temp. | 140° C. |
| Aspirator Setting | 100% (.38 mbar) |
| Airflow rate | 800 NL/h |
| Pump Setting | 10% (145 ml/hr) |
| Outlet reading | 78° C. |

Produced in a Buchi 191 Spray Drying Apparatus

The particles formed were spherical with 74% below 5 microns and 99% below 10 microns, as can be seen in the photo micrographs of FIGS. 1 and 2, FIG. 2 showing particles obtained according to Example 1.

### Example 2

Particles produced with PVP by Spray-Drying

PVP grade K30 used

| | |
|---|---|
| Solution concentration | 1.5% w/v in 95% ethanol, |
| Inlet temperature | 84° C. |
| Aspirator setting | 100% (−38 mbar) |
| Airflow rate | 800 Nl/hr |
| Pump setting | 15% (220 ml/hr) |
| Outlet reading | 60-61° C. |

Photomicrographs of initial PVP and particles produced. The particles formed were spherical with 95% of particles below 5 micron and 100% below 8 microns, as can be seen in the photo micrographs of FIGS. 3 and 4, FIG. 4 showing particles obtained according to Example 2.

One or more actives can be dissolved or suspended in Examples 1 or 2 to give suitable particles for inhalation

What is claim is:

1. Particles for drug delivery by inhalation, comprising at least one active ingredient which is non-crystalline.

2. Particles according to claim 1, further comprising a second or more active(s) and/or one or more excipient.

3. Particles for drug delivery according to claim 1 or claim 2, the particles containing a plurality of non-crystalline active ingredients.

4. Particles according to claim 3, further comprising an outer surface of the particles being substantially smooth.

5. Particles according to claim 3, particles being substantially spherical.

6. Particles according to claim 5, particles being oblate spheroidal.

7. Particles according to claim 3, particles being substantially oval.

8. Particles according to claim 3, particles being substantially elliptical.

9. Particles according to any preceding claim, having a particle size in the range 0.5 μm to 5 μm.

10. Particles according to claim 9, the particle size being between 1 μm to 3 μm.

11. Particles according to claim 9 when dependent on claim 7 or claim 8, the longer axis of an oval or elliptical particle having a length between 1 μm to 3 μm.

12. Particles according to any preceding claim, the particles being electrically uncharged.

13. Particles according to any preceding claim, provided by a method selected from the group comprising rapid expansion of supercritical solutions, precipitation from gas saturated solutions, gas anti-solvent systems, aerosol solvent extraction systems and spray drying processes.

14. Particles according to any preceding claim there being from two to four active ingredients.

15. Particles according to any preceding claim, comprising a pharmaceutically acceptable particular excipient or excipient.

16. Particles according to any preceding claim, the active ingredients comprising a: β₂-agonist and a steroid.

17. Particles according to claim 15, comprising fluticasone-dipropionate and salmeterol xinafoate.

18. Particles according to any one of claims 1 to 15, comprising formoterol and budesonide.

19. Particles for drug delivery according to any of claims 2 to 18, the or each excipient being soluble in conditions obtaining in the nose, lung(s) or mouth of a human or animal.

20. An inhalation composition, comprising particles which incorporate at least one active ingredient which is non-crystalline.

21. A composition according to claim 20, further comprising a second or more active(s) and/or one or more excipient.

22. A composition according to claim 20 or 21, the particles containing a plurality of active ingredients, which active ingredients are non-crystalline.

23. A composition according to claims 20 to 22, there being from two to four active ingredients.

24. A composition according to any of claims 21 to 23, the particles comprising a pharmaceutically acceptable excipient within the particle.

25. A composition according to any of claims 21 to 24, the particles comprising a pharmaceutically acceptable excipient or excipient where a main excipient is in a greater proportion than the active or actives.

26. A composition according to claim 25, the main excipient being Mannitol or PVP.

27. A composition according to any of claims 21 to 26, comprising one or more additional carrier excipient(s).

28. A composition according to claim 27, said excipient(s) comprising a modifier or stabiliser.

29. A composition according to claim 27, said excipient(s) comprising a chemical buffer, antioxidant and the like.

30. A composition according to claim 27, said excipient(s) comprising a surface modifier or surfactant.

31. A composition according to any of claims 20 to 30, the outer surface of the particles being substantially smooth.

32. A composition according to claim 31, the particles being substantially spherical.

33. A composition according to claim 32, the particles being oblate spheroidal.

34. A composition according to claim 31, the particles being substantially oval.

35. A composition according to claim 31, the particles being substantially elliptical.

36. A composition according to any of claims 20 to 35, the particles thereof having a particle size in the range 0.5 μm-5 μm.

37. A composition according to claim 36, having a particle size of 1μ to 3μ.

38. A composition according to claim 37, the particles being electrically uncharged.

39. A composition according to any of claims 20 to 38, provided by a method selected from the group comprising rapid expansion of supercritical solutions, precipitation from gas saturated solutions, gas anti-solvent systems, aerosol solvent extraction systems, and a spray drying process.

40. A composition according to any of claims 20 to 39, comprising fluticasone and salmeterol xinafoate as active ingredients.

41. A composition according to any of claims 20 to 39, the particles comprising formoterol and budesonide as active ingredients.

42. A composition according to any of claims 29 to 39, the particles containing one or more cannabinoids as an active ingredient.

43. A composition according to claim 42, the cannabinoid comprising delta-8 or delta-9 tetrahydrocannabinol.

44. An inhaler device, comprising an inhalation composition according to any of claims 20 to 43.

45. A pulmonary nasal inhalation device, comprising an inhalation composition according to any of claims 20 to 44.

46. A device according to claim 44 or claim 45, the rain excipient being non-soluble in the propellant or propellants.
